(19) United States
(12) Patent Application Publication
(10) Pub. No.: US 2008/0206826 A1
(43) Pub. Date: Aug. 28, 2008

(54) Method for Producing Single Enantiomer Epoxides by the Adh Reduction of a-Leaving Group-Substituted Ketones and Cyclization

(76) Inventors: Andreas Meudt, Hofheim (DE); Richard Wisdom, Eppstein (DE); Claudius Boehm, Frankfurt am Main (DE)

Correspondence Address: PROPAT, L.L.C., 425-C SOUTH SHARON AMITY ROAD, CHARLOTTE, NC, 28211-2841, US

(21) Appl. No.: 11/917,777

(22) PCT Filed: Jun. 7, 2006
(86) PCT No.: PCT/EP2006/005437

§ 371 Date: Dec. 17, 2007

(30) Foreign Application Priority Data
Jun. 18, 2005 (DE) 10 2005 028 312.8

(51) Int. Cl. C12P 17/02

(52) U.S. Cl. 435/123

(57) ABSTRACT

The invention relates to a method for producing single enantiomer epoxides by reducing α-leaving group-substituted ketones with (R)- or (S)-selective alcohol dehydrogenases in the presence of a cofactor and optionally a suitable system for regenerating the oxidised cofactor, to produce the corresponding single enantiomer alcohols and subsequently, by means of cyclisation induced by a base, the corresponding single enantiomer epoxides (EQUATION 1)

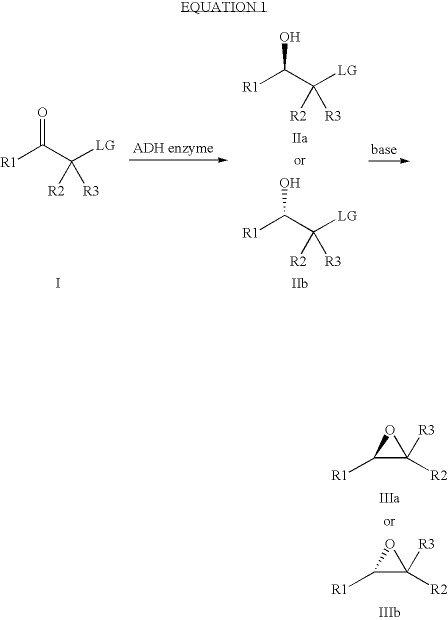

wherein LG may stand for F, Cl, Br, I, OSO₂Ar, OSO₂CH₃, OSO₂R or OP(O)OR₂, and R₁, R₂ and R₃, independently of one another, stand for hydrogen, a branched or unbranched, optionally substituted C₁-C₂₀-alkyl radical, symbolize an optionally randomly substituted C₃-C₁₋₁₀-cycloalkyl or alkenyl radical or a randomly substituted carbo- or heterocyclic aryl radical, or correspond to a radical from the group CO₂R, CONR₂, COSR, CS₂R, C(NH)NR2, CN, CHaI₃, ArO, ArS, RO, RS, CHO, OH, NHR, NR₂, Cl, F, Br, I or SiR₃.

Method for producing single enantiomer epoxides by the ADH reduction of α-leaving group-substituted ketones and cyclization

The invention relates to a process for preparing enantiomerically pure epoxides by (R)- or (S)-alcohol dehydrogenase reduction of α-leaving group-substituted ketones to the corresponding enantiomerically pure alcohols and subsequent base-induced cyclization to the corresponding enantiomerically pure epoxides (EQUATION 1).

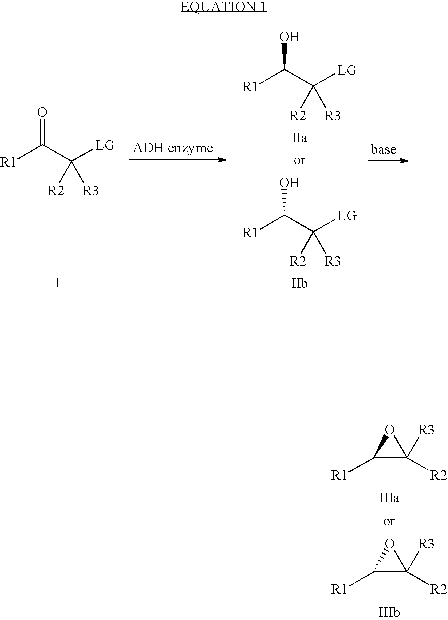

The proportion of enantiomerically pure compounds in the overall market for pharmaceutical fine chemicals and precursors was already over 40% in 2004 and is growing at high speed. Enzymatic applications in particular are notable for the highest growth rates in overall organic synthesis; according to the study, up to 35% annual growth up to 2010 is forecast. On an almost daily basis, new interesting descriptions are appearing for the preparation of enantiomerically pure intermediates of a wide variety of different substance classes. It is all the more astonishing that there are only a few generally applicable methods for preparing enantiomerically pure epoxides, in particular since these strained three-membered ether rings are usable in an extremely versatile manner in organic synthesis. The most frequently employed method is the destruction of the undesired enantiomer by transition metal catalysis or by enzymatic catalysis and subsequent isolation of the desired enantiomer in pure form. The great, disadvantage of this method is the loss of at least 50% of the amount of substrate by the necessary destruction of the incorrect enantiomer. Combined with further process problems, resulting yields are often only 40% and worse.

Catalytic enantioselective chemical standard methods for the enantioselective reduction of ketones are asymmetric hydrogenation with homogeneous noble metal catalysts, reduction by means of organoboranes [H. C. Brown, G. G. Pai, J. Org. Chem. 1983, 48, 1784;], which are prepared from borohydrides and chiral diols or amino alcohols [K. Soai, T. Yamanoi, H. Hikima, J. Organomet. Chem. 1985, 290; H. C. Brown, B. T. Cho, W. S. Park, J. Org. Chem. 1987, 52, 4020], reduction by means of reagents prepared from borane and amino alcohols [S. Itsuno, M. Nakano, K. Miyazaki, H. Masuda, K. Ito, H. Akira, S, Nakahama, J. Chem. Soc., Perkin Trans 1, 1985, 2039; S. Itsuno, M. Nakano, K. Ito, A. Hirao, M. Owa, N. Kanda, S, Nakahama, ibid. 1985, 2615; A. K. Mandal, T. G. Kasar, S. W. Mahajan, D. G. Jawalkar, Synth. Commun. 1987, 17, 563], or by means of oxazaborolidines [E. J. Corey, R. K. Bakshi, S. Shibata, J. Am. Chem. Soc. 1987, 109, 5551; E. J. Corey, S. Shibata, R. K. Bakshi, J. Org. Chem. 1988, 53, 2861]. The great disadvantages of these methods are the use of expensive chiral auxiliaries which often have to be prepared by complicated synthesis, the use of hydrides which can release explosive gases, and the use of heavy metals, which often contaminate the resulting product and are difficult to remove.

The catalytic enantioselective biochemical standard methods for preparing the enantiomerically pure epoxides utilize baker's yeast (Saccharomyces cerevisiae) in a fermentation method [M. de Carvalho, M. T. Okamoto, P. J. S. Moran, J. A. R. Rodrigues, Tetrahedron 1991, 47, 2073] or other microorganisms [EP 0 198 440 B1] in the so-called “whole-cell method”, Cryptococcus macerans [M. Imuta, K. I. Kawai, H. Ziffer, J. Org. Chem. 1980, 45, 3352], or a combination of NADH2 and horse liver ADH [D. D. Tanner, A. R. Stein, J. Org. Chem. 1988, 53, 1642].

Especially the potential contamination of the products with animal pathogens, as, for example, in the latter-case, often prevents even the application of such methods in the preparation of precursors for the pharmaceutical industry.

A further great disadvantage of whole cell methods in particular is the complicated workup of fermentation solutions to isolate the desired products. In particular, though, the literature discusses the problem that cells usually comprise more than one ketoreductase which additionally often have different enantioselectivities, such that poor ee values are obtained overall.

It would therefore be very desirable to have an enzymatic process which, proceeding from readily available α-leaving group-substituted ketones to the corresponding enantiomerically pure alcohols and subsequent base-induced cyclization, affords the corresponding enantiomerically pure epoxides in a theoretical yield of 100%. In addition, the corresponding methodology should make both enantiomers obtainable in principle. On the basis of the known and already discussed problems in the case of use of whole cells, isolated alcohol dehydrogenases, which have only recently become sufficiently available, should additionally be used.

The present process solves all of these problems and relates to a process for preparing enantiomerically pure epoxides by reduction of α-leaving group-substituted ketones with an (R)- or (S)-alcohol dehydrogenase (ADH) enzyme in the presence of a cofactor and optionally of a suitable system for regenerating the oxidized cofactor to the corresponding enantiomerically pure alcohols and subsequent base-induced cyclization to the corresponding enantiomerically pure epoxides (EQUATION 1), in which

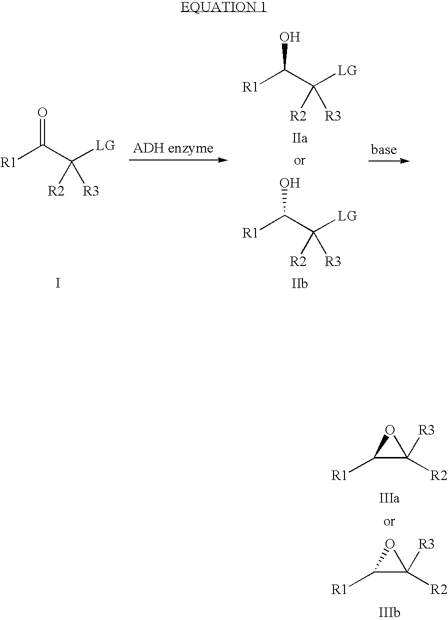

R₁, R₂ and R₃ each independently represent hydrogen, halogen, a branched or unbranched, optionally substituted C₁-C₂₀-alkyl radical, a C₃-C₁₀-cycloalkyl radical which may have any substitution, alkenyl radical or a carbo- or heterocyclic aryl radical which may have any substitution, or a radical from the group of CO₂R, CONR₂, COSR, CS₂R, C(NH)NR₂, CN, CHal₃, ArO, ArS, RO, RS, CHO, OH, NH₂, NHR, NR₂, Cl, F, Br, I or SiR₃, and LG may be F, Cl, Br, I, OSO₂Ar, OSO₂CH₃, OSO₂R or OP(O)OR₂.

Suitable ADH enzymes are (R)- or (S)-alcohol dehydrogenases. Preference is given to using isolated (cell-free) ADH enzymes having an enzyme activity of from 0.2 to 200 kU per mole of substrate, more preferably from 0.5 to 100 kU of enzyme activity per mole of substrate, most preferably from 1 to 50 kU of enzyme activity per mole of substrate.

Preference is given to using the enzyme in catalytic to superstoichiometric amounts in relation to the starting compound.

Suitable cofactors are NADPH₂, NADH₂, NAD or NADP, particular preference being given to using NAD or NADP. Preference is given to a loading with from 0.1 to 10 g of cofactor per 10 mol of substrate, particular preference to from 0.5 to 1.5 g of cofactor per 10 mol of substrate. Preference is given to performing the process according to the invention in such a way that it is conducted in the presence of a suitable system for regenerating the oxidizing cofactor which is recycled continuously during the process. For the reactivation of the oxidized cofactors, typically enzymatic methods or other methods known to those skilled in the art are used.

For example, the cofactor is recycled continuously by coupling the reduction with the oxidation of isopropanol to acetone with ADH, and can thus be used in several oxidation/reduction cycles.

Another commonly used method is the use of a second enzyme system in the reactor. Two methods described in detail are, for example, the use of formate dehydrogenase for oxidation of formic acid to carbon dioxide, or the use of glucose dehydrogenase to oxidize glucose, to name just a few.

In a preferred embodiment, the reaction is performed in a solvent. Suitable solvents for the ADH reduction are those which do not give rise to any side reactions; these are organic solvents, for example methanol, ethanol, isopropanol, linear and branched alcohols, ligroin, butane, pentane, hexane, heptane, octane, cyclopentane, cyclohexane, cycloheptane, cyclooctane, dichloromethane, chloroform, carbon tetrachloride, 1,2-dichloroethane, 1,1,2,2-tetrachloroethane, methyl acetate, ethyl acetate, propyl acetate, butyl acetate, dimethylformamide, diethylformamide, dimethylacetamide, diethylacetamide, diethyl ether, diisopropyl ether, tert-butyl methyl ether, THF, dioxane, acetonitrile or mixtures thereof. Preference is given to using linear or branched alcohols or linear, branched or cyclic ethers, for example methanol, ethanol, isopropanol, diisopropyl ether, tert-butyl methyl ether, tetrahydrofuran (THF), dioxane or mixtures thereof; very particular preference is given to using ethanol, isopropanol, linear and branched alcohols, diethyl ether, diisopropyl ether, tert-butyl methyl ether, THF, dioxane or mixtures thereof. In a further preferred embodiment, the process can also be performed without addition of solvent.

In some cases, it is advisable to add a buffer to the reaction solution in order to stabilize the pH and to be certain that the enzyme can react in the pH range optimal for it. The optimal pH range is different from enzyme to enzyme and is typically in the range from pH 3 to 11. Suitable buffer systems are known to those skilled in the art, so that there is no need to discuss them further at this point.

The reduction to the alcohols (IIa) or (lib) can generally be performed at temperatures in the range from −100 to +120° C.; preference is given to temperatures in the range from −30 to +50° C., particular preference to temperatures in the range from 0 to +40° C., lower temperatures generally correlating with higher selectivities. The reaction time depends on the temperature employed and is generally from 1 to 72 hours, especially from 4 to 45 hours.

The ee values of the alcohols obtained as intermediates are significantly > 95% ee, in most cases > 99%, with simultaneously very high tolerance toward functional groups in the substrate.

The cyclization of the alcohols (IIa) or (IIb) to the epoxides can be performed generally at temperatures in the range from −100 to +120° C.; preference is given to temperatures in the range from −30 to +50° C., particular preference to temperatures in the range from 0 to +40° C. The reaction time depends on the temperature employed and is generally from 1 to 72 hours, especially from 24 to 60 hours. Sufficient conversion can be ensured, for example, by GC or HPLC reaction monitoring. Preference is given to adjusting the temperature of the reaction solution to the reaction temperature before the ADH enzyme is added.

Suitable bases for the cyclization are in principle all bases. Preference is given to amine bases, carbonates, hydrogencarbonates, hydroxides, hydrides, alkoxides, phosphates, hydrogenphosphates, more preferably tertiary amines, most preferably sodium hydroxide, potassium hydroxide, triethylamine or pyridine.

Preference is given to using the base in a stoichiometric or superstoichiometric amount in relation to the compound (IIa) or (lib).

The isolation of the products is preferably undertaken either by distillation or by crystallization. In general, as a result of the properties of the enzymes, the ee values are significantly greater than 99%, as a result of which no further purification is required.

The substrate breadth of this novel technology is very high. It is just as possible to use α-leaving group-substituted ketones with aryl radicals of different substitution pattern as it is to use aliphatic halomethyl ketones. Chloroacetyl ketones react here in particularly good yields and high ee values.

The novel process thus affords a wide range of enantiomerically pure epoxides in very high yields of > 85%, usually > 90%, and very high ee values, and it is possible to obtain both enantiomers depending on the enzyme used.

The process according to the invention will be illustrated by the examples which follow without restricting the invention thereto:

### EXAMPLE 1

### (S)-4-fluorophenyloxirane

A mixture of 150 ml of sodium phosphate buffer (0.1 M, pH 7.0), 22.2 g of 2′-chloro-4-fluoroacetophenone, 60 ml of isopropanol, 50 ml of diisopropyl ether, 30 mg of NADP disodium salt and 2750 U Lactobacillus brevis alcohol dehydrogenase (Jülich Fine Chemicals) was stirred at 20° C. for 64 hours. Reaction monitoring showed a conversion of 95%. 20 ml of sodium hydroxide solution (10 M) were added to the solution which was stirred for a further 2 hours. Reaction monitoring indicated complete conversion of the alcohol to the epoxide. 2 g of Celite Hyflo were added to this reaction mixture which was filtered, and the filtrate was subsequently extracted with methyl tert-butyl ether (MTBE). The organic extracts were distilled. 13.8 g of product were isolated (yield 92%, ee> 99%, chiral GC (cyclodextrin β, BetaDex-Supelco), purity 99% (GC a/a)).

### EXAMPLE 2

### (R)-3-chlorophenyloxirane

A mixture of 1 ml of sodium phosphate buffer (0.1 M, pH 7.0), 240 mg of magnesium sulfate, 46 mg of 2′-chloro-3-chloroacetophenone, 270 μl of isopropanol, 300 μl of diisopropyl ether, 0.5 mg of NADP disodium salt and 20 U Rhodococcus spec. ADH was stirred at 20° C. for 30 hours. Reaction monitoring showed a conversion of > 90%. 2 ml of sodium hydroxide solution (10 M) were added to this solution which was stirred for a further 2 hours. Reaction monitoring indicated complete conversion of the alcohol to the epoxide (chiral GC (cyclodextrin β, BetaDex-Supelco)> 99% ee). GC yield 92% (a/a).

### EXAMPLES 3 TO 5

In the same way as described above, it was possible to obtain the following oxiranes:

| | GC yield | ee/% |
|---|---|---|
| (S)-3-chlorophenyloxirane | 92% | >99 |
| (R)-4-chlorophenyloxirane | 93% | >99 |
| (R)-2-chlorophenyloxirane | 88% | >98.5 |

1. A process for preparing enantiomerically pure epoxides comprising reducing α-leaving group-substituted ketones with (R)- or (S)-selective alcohol dehydrogenases in the presence of a cofactor and optionally of a suitable system for regenerating the oxidized cofactor to the corresponding enantiomerically pure alcohols and subsequently base-inducing cyclization to the corresponding enantiomerically pure epoxides (EQUATION 1), in which 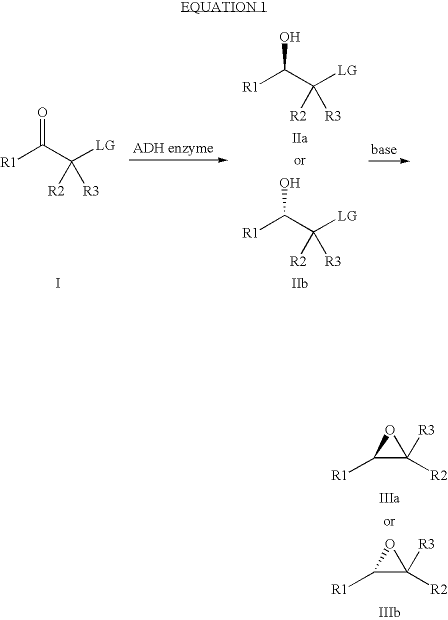
LG may be F, Cl, Br, I, OSO₂Ar, OSO₂CH₃, OSO₂R or OP(O)OR₂ and
R₁, R₂ and R₃ each independently represent hydrogen, a branched or unbranched, optionally substituted C₁-C₂₀-alkyl radical, a C₃-C₁₀-cycloalkyl radical which may have any substitution, alkenyl radical or a carbo- or heterocyclic aryl radical which may have any substitution, or a radical from the group of CO₂R, CONR₂, COSR, CS₂R, C(NH)NR₂, CN, CHal₃, ArO, ArS, RO, RS, CHO, OH, NHR, NR₂, Cl, F, Br, I or SiR₃.

2. The process as claimed in claim 1, wherein the α-leaving group-substituted ketones are reduced by using isolated (cell-free) ADH enzymes.

3. The process as claimed in claim 1, wherein the (R)- or (S)-alcohol dehydrogenases have an enzyme activity of from 0.2 to 200 kU per mole of substrate.

4. The process as claimed in claim 1, wherein the enzymatic reduction is performed in the presence of a cofactor selected from for example NADPH₂, NADH₂, NAD or NADP.

5. The process as claimed in claim 1, wherein the oxidized cofactor is reduced by systems and is recycled.

6. The process as claimed in claim 1, wherein LG is F or Cl.

7. The process as claimed in claim 1, wherein the process is performed in an organic solvent.

8. The process as claimed in claim 1, wherein the reduction and the subsequent cyclization are performed at from −100 to +120° C.

9. The process as claimed in claim 1, wherein the ee values of the alcohols obtained as intermediates and of the epoxides are > 95% ee.

10. The process as claimed in claim 1, wherein the base used for the cyclization is selected from amine bases, carbonates, hydrogencarbonates, hydroxides, hydrides, alkoxides, phosphates and hydrogenphosphates.

11. The process as claimed in claim 1, wherein the temperature of the reducing solution is adjusted to the reducing temperature before the ADH enzyme is added.

12. The process as claimed in claim 1, wherein the dehydrogenase is used in a catalytic to superstoichiometric amount in relation to the α-leaving group-substituted ketones.

13. The process as claimed at least one of the preceding claim 1, wherein the process further comprises isolating the products.

14. The process as claimed claim 13, wherein the isolating step comprises distillation or crystallization.
